(19) United States
(12) Patent Application Publication
(10) Pub. No.: US 2024/0216579 A1
(43) Pub. Date: Jul. 4, 2024

(54) DESCEMET MEMBRANE ENDOTHELIAL KERATOPLASTY (DMEK) ASSEMBLIES AND INJECTORS WITH FRICTION-REDUCING COATINGS

(71) Applicant: REGENTS OF THE UNIVERSITY OF MINNESOTA, Minneapolis, MN (US)

(72) Inventors: Joshua Honghan Hou, Minneapolis, MN (US); Peter Bedard, St. Paul, MN (US); Chun Wang, Minneapolis, MN (US); Ching Yuan, Maple Grove, MN (US); Sung Lee, St. Paul, MN (US); Aaron Bae, Edina, MN (US)

(21) Appl. No.: 18/558,837

(22) PCT Filed: May 13, 2022
(86) PCT No.: PCT/US2022/029249

§ 371 Date: Nov. 3, 2023

Related U.S. Application Data
(60) Provisional application No. 63/188,217, filed on May 13, 2021.

(51) Int. Cl. A61L 27/36; A61F 2/14; A61L 27/34; A61L 27/38; A61L 27/54

(52) U.S. Cl. CPC A61L 27/3604; A61F 2/142; A61F 2/148; A61L 27/34; A61L 27/3641; A61L 27/3808; A61L 27/3839; A61L 27/54; A61L 2300/404; A61L 2400/06; A61L 2400/10; A61L 2400/12; A61L 2420/02; A61L 2420/06; A61L 2430/16

(57) ABSTRACT

A graft injector assembly includes a coating disposed on an interior surface of an injector body to form a lumen. The lumen is structured to store a graft comprising corneal tissue and to allow the graft to exit distally from the injector body, preferably with minimal or no damage to the corneal tissue. The corneal tissue may include a Descemet membrane and endothelial cells, which may be used in Descemet Membrane Endothelial Keratoplasty (DMEK). In one example, the assembly includes an organosilane coating disposed on a glass injector body.

### CROSS-REFERENCE TO RELATED APPLICATION

This application claims the benefit of U.S. Provisional Patent Application No. 63/188,217, filed May 13, 2021, which is incorporated herein by reference in its entirety.

### SUMMARY

This disclosure provides devices and techniques that generally relate to reducing trauma to endothelial cells in a DMEK graft that results from contact with a surgical injector device. Various embodiments involve a non-adhesive organosilane coating on the injector to reduce friction between the graft and an interior surface of the injector device.

Thus, in one aspect, this disclosure describes a graft injector assembly that includes an elongated injector body with an interior surface and a coating disposed on the interior surface of the injector body.

In some embodiments, the coating reduces adhesion of corneal tissue to the injector body compared to an injector body without the coating.

In some embodiments, the coating includes a material that is more hydrophilic than the material that forms the injector body.

In some embodiments, the assembly includes a corneal graft. In some of these embodiments, the corneal graft includes a Descemet membrane and endothelial cells.

In some embodiments, the elongated injector body is formed from a glass material.

In some embodiments, the coating is disposed on the entire interior surface of the injector body.

In some embodiments, the coating is covalently bonded to the body.

In some embodiments, the coating includes material that includes a positively charged functional group, a charge-neutral functional group, or both.

In some embodiments, the coating includes an organosilane material.

In some embodiments, the coating includes a nanoparticulate material.

In some embodiments, the coating includes a functional bioactive moiety compatible with the graft that lubricates the interior surface. In some of these embodiments, the functional bioactive moiety includes an amine, a carboxylic acid, a hydroxyl, a sugar, a polysaccharide, a glycosaminoglycan, a peptide, a protein, a polymer. In embodiments in which the functional bioactive moiety includes a polymer, the polymer can include a polyethylene glycol, a polyvinyl alcohol, a polyhydroxy ethyl methacrylate, a poly(meth)acrylic acid, a polyvinylpyrrolidone, or any combination of these.

In some embodiments, the coating includes a polymer that includes an amine, polyethylene glycol, or both.

In some embodiments, the coating includes polymers of various chain lengths.

In some embodiments, the coating further includes an antimicrobial agent. In some of these embodiments, the antimicrobial agent includes an antifungal agent.

In another aspect, this disclosure describes a method that generally includes bonding a coating to an interior surface of a graft injector, loading a solution into the lumen of the graft injector, and loading a graft into the lumen of the graft injector.

In yet another aspect, this disclosure describes a method that includes bonding a coating to an interior surface of an elongated injector body and attaching a functional bioactive moiety to the coating. In some embodiments, the bioactive functional moiety reduces adhesion between the injector body and a corneal tissue graft compared to adhesion between a corneal tissue graft and the interior surface of the injector body without the functional bioactive moiety.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1 is a conceptual diagram that illustrates one example of an environment for using a graft injector assembly having a coating according to techniques of the present disclosure, which can be used, for example, to anatomically replace diseased or damaged Descemet membrane and endothelium.

FIGS. 2A-D illustrate one example of loading of a graft into a graft injector of the graft injector assembly of FIG. 1 and, in particular, (A) an overhead view of the graft outside of the graft injector, (B) a side view of one example of a graft, (C) a side view of one example of a graft loaded into the graft injector, and (D) a side view of another example of a graft loaded into the graft injector.

FIG. 3 is a conceptual diagram that illustrates one example of a coating method for use with the graft injector of FIG. 2.

FIG. 4 is a conceptual diagram that illustrates another example of a coating method for the graft injector of FIG. 2 to attach a functional bioactive moiety.

FIG. 5 is a conceptual diagram that illustrates yet another example of a coating method for the graft injector of FIG. 2 to attach an antifungal agent.

FIGS. 6A-E illustrate one example of a method of placing a graft using the graft injector assembly of FIG. 1 in Descemet membrane endothelial keratoplasty (DMEK) and, in particular, (A) a perspective view of injecting the graft, (B) a cutaway view of using jets of fluid to facilitate unfurling the graft, (C) a cutaway view of tapping the cornea to facilitate unfurling the graft, (D) a cutaway view of centering the graft, and (E) positioning the graft against the cornea using, for example, insertion of a gas.

FIGS. 7A-B are vital dye staining images that illustrate (A) a graft before loading and (B) a graft after loading and ejection from a graft injector without a coating.

FIG. 8 is a conceptual diagram of one example of an apparatus to test lubricity of modified glass surfaces for use with the graft injector of FIG. 2.

FIG. 9 is an illustration of one example of raw images of stained cells and images segmented into live, dead, and background areas with ImageJ Weka Segmentation plug-in for use in evaluating endothelial cell loss with the graft injector assembly of FIG. 1.

FIG. 10 is a bar graph demonstrating the decrease in friction between two glass surfaces treated with different molecular weights of polyethylene glycol (PEG) as measured using an apparatus as in FIG. 8. A tensile testing machine was used to test how much force it took to pull two overlapping glass slides apart. Each grip on the machine held a coated glass slide, with heights adjusted such that the coated regions overlapped over a flat, fixed area. Water was placed between the coated slides before overlapping them since in order to test friction under aqueous conditions. The force readouts were used as relative measurements of friction. The 20k PEG demonstrated the lowest frictional force (in Newtons).

FIG. 11 is a bar graph demonstrating the decrease in water contact angle with polyethylene glycol (PEG) modification of a glass slide. Water contact angle was measured as the angle between the glass and the tangential of the water surface right at the edge of a water droplet. The 20k PEG demonstrated the lowest water contact angle and thus greatest hydrophilicity.

FIG. 12 is a conceptual diagram demonstrating the method for testing endothelial cell viability, which is proportional to the density of endothelial cells on a fixed-sized DMEK graft, while the graft was inside the injector and after ejection of the graft. This method, which employs ALAMARBLUE (Trek Diagnostic Systems LLC, Cleveland, OH) as an indicator of cell viability, was used to isolate the endothelial cell loss from ejection of the donor graft from a glass injector.

FIG. 13 is a bar graph demonstrating the statistically significant difference in loss of ALAMARBLUE (Trek Diagnostic Systems LLC, Cleveland, OH) fluorescence after ejection of a DMEK graft. The loss in ALAMARBLUE fluorescence signaling is proportional to endothelial cell loss. Ejecting a DMEK graft from an uncoated injector resulted in 38.0% loss of ALAMARBLUE fluorescence, while ejecting a DMEK graft from a coated injected resulted in a 29.5% loss of ALAMARBLUE signal (N=9; P=0.0446).

FIG. 14 is a bar graph demonstrating the difference in the internal and external bore sizes of the glass injectors used in the testing shown in FIG. 13. The bore sizes of the injectors were measure at the internal and external openings to ensure that the glass injectors were similar in size and to show that variability in bore diameter (narrow versus wide bore openings) did not contribute to the difference in endothelial cell loss shown in FIG. 13. There was no statistically significant difference in the internal and external bore diameters.

FIG. 15 is a line graph demonstrating how both ALAMARBLUE (Trek Diagnostic Systems LLC, Cleveland, OH) fluorescence and calcienAM correlate with endothelial cell density. Endothelial cells were cultured and seeded at known cell densities (500 cells/mm², 1000 cells/mm², 2000 cells/mm², 3000 cells/mm²). Cells at each density were incubated with ALAMARBLUE for one hour at 37° C. and the fluorescence signal measured using a microplate reader (BioTek Synergy, Agilent Technologies, Inc., Santa Clara, CA). Additional cells at each density were stained with calcienAM. Cells were then lysed to release the intracellular dye. Samples were then dilated 1:10 and the fluorescence signal from calcienAM was then measured using a microplate reader (BioTek Synergy, Agilent Technologies, Inc., Santa Clara, CA). Both the ALAMARBLUE and calcienAM fluorescence demonstrated a strong linear correlation with endothelial cell density.

### DETAILED DESCRIPTION OF ILLUSTRATIVE EMBODIMENTS

The present technology is generally related to injectors and, in particular, injectors for Descemet Membrane Endothelial Keratoplasty (DMEK).

A common indication for corneal transplantation is endothelial failure. Endothelial cells are specialized cells found on the posterior surface of the cornea that actively pump fluid out of the cornea, keeping the corneal from swelling. Loss of endothelial cells can lead to swelling and opacification of the cornea. This may result in loss of vision and, in severe cases, painful blister (bullae) formation on the surface of the eye. Because endothelial cells do not regenerate, one of the only viable treatments for endothelial failure is transplantation of more endothelial cells.

Descemet Membrane Endothelial Keratoplasty (DMEK) is a type of endothelial cell transplant surgery for endothelial failure. In DMEK, the damaged endothelial cells and their basement membrane (which is called Descemet membrane) are stripped from the back of the patient's cornea. After that, the Descemet membrane and healthy endothelial cells from a cadaveric donor cornea are transplanted into the patient's eye.

Success of DMEK can depend heavily on the amount of endothelial cell loss (ECL) that occurs during preparation and transplantation processes. Injectors have been used to deliver the Descemet membrane and healthy endothelial cells to the patient's eye. Glass injectors have largely supplanted plastic injectors due to the lower risk of ECL with glass since adhesion and friction of cells against glass is less than for commercial plastics used in intraocular surgery. However, using glass injectors can still result in up to a 27% decrease in endothelial cell density.

Use of preloaded Descemet membrane endothelial keratoplasty (DMEK) grafts is growing in popularity among corneal surgeons. The convenience of having the graft already loaded and ready for insertion at the time of surgery not only simplifies the surgical procedure, but also reduces overall surgical time. Further, preloading a graft can reduce the risk of intraoperative complications associated with loading grafts. One challenge in using preloaded grafts, however, is the risk of endothelial damage and contamination during preparation and transportation of preloaded grafts. For example, there can be more endothelial cell loss (ECL) in preloaded DMEK grafts shipped with a typical endothelium-out scroll conformation than in grafts shipped with an atypical endothelial-in tri-fold conformation.

As noted above, success of DMEK can depend heavily on the number of endothelial cells that survive the preparation and transplantation process. Excessive injury to donor endothelial cells may result in slower visual recovery, increased risk of graft detachment from the patient's cornea, and increased risk of primary graft failure. In DMEK, significant endothelial cell loss (ECL) can occur during the loading, transportation, and insertion of donor grafts using standard insertion devices, such as injectors made of glass. This is especially significant in practices involving DMEK grafts being prepared in the eye bank and shipped to surgeons already loaded into an injector, which may be described as a preloaded DMEK graft or assembly. Expanded surgeon adoption of preloaded DMEK assemblies has led to even more glass-to-cell contact time for DMEK grafts, since the time in the DMEK graft spends in the injector may be dramatically longer compared to when surgeons load the DMEK grafts themselves at the time of surgery.

Preloaded DMEK assemblies can still result in endothelial cell loss (ECL) due to one or more of preparation, loading, transportation, insertion of DMEK grafts, peeling, trephining, loading, and/or ejecting a DMEK. Given the expanding surgeon interest in preloaded DMEK assemblies, minimizing this endothelial damage may facilitate optimizing patient outcomes.

Currently, the endothelial cell loss is simply tolerated by surgeons and eye banks, even though it is not ideal for patients. Post-operatively, there can be up to an additional 48% loss in endothelial cell density after transplantation. Because this post-operative loss is largely assumed to be unavoidable, a standard practice has been to use donor corneas with high enough pre-operative endothelial cell density that they still work post-operatively after factoring in the loss. The minimum endothelial cell density that a DMEK graft should have pre-operatively in order to be transplantable in some cases is 2000 cells/mm². The approximate threshold at which a graft will fail and no longer function clinically in some cases is 500 cells/mm². Losing 48% in endothelial cell density may not be catastrophic enough to cause clinical graft failure (for example, less than 500 cells/mm²) if the starting endothelial cell density is high enough. However, high endothelial cell density corneas are not always available. Furthermore, because cells are slowly lost over the lifetime of the transplant, even after surgery, the more cells that survive the transplantation process, the longer the graft survives.

Some of the post-operative cell loss seen with DMEK may be from intraoperative trauma as the graft is oriented and positioned in the eye. However, pre-operative decreases in endothelial cell density can be attributed to loading, transporting, and ejecting a DMEK graft from a standard glass DMEK injector. This cell loss may be largely due to mechanical trauma of the endothelial cells as they rub or collide with the lumen walls of the DMEK injector. In particular, abrasive contact or adhesion between endothelial cells and the wall of standard glass inserters may be described as the primary cause of ECL in preloaded DMEK in some cases.

This disclosure provides techniques that generally relate to reducing trauma to the graft when in contact with the injector, such as significant ECL in preloaded DMEK grafts due to contact between endothelium and the lumen wall of the injector. Various aspects create a reduced friction, non-adhesive organosilane coating on the injector.

The present disclosure addresses various challenges, such as preventing damage that appears to occur during loading and transportation of the tissue and likely results from contact between the endothelium and the wall of an injector, such as a modified Jones tube.

Surface coating technology provides a novel solution to these challenges. In one aspect, the present disclosure provides a graft injector assembly that includes an interior coating. The graft injector may reduce the trauma to the graft when in contact with the injector body. Various suitable coatings may be used on the interior surface of the injector body to reduce friction between the graft injector assembly and the graft, which is formed of corneal tissue or cellular material. For example, a non-adhesive organosilane coating may be bonded to the interior surface of a glass injector body. Existing types of injectors, such as simple glass injectors or preloaded DMEKs, may be provided with the interior coating. In other words, injectors having various shapes may all benefit from applying the interior coating to at least their interior surface.

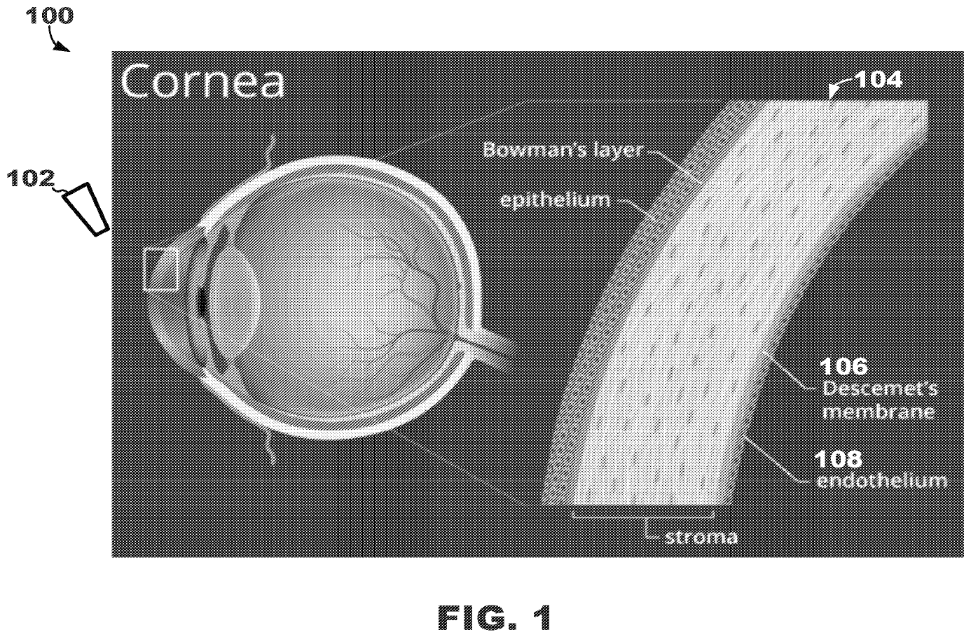

FIG. 1 shows one example of an environment 100 for use with a graft injector assembly 102 having a coating according to techniques of the present disclosure. As illustrated, the environment 100 includes a cornea 104 of the patient's eye. The cornea 104 is the clear shield in front of the eye. The cornea 104 has several layers. The cornea 104 includes a Descemet membrane 106 and an endothelium 108 that covers Descemet membrane, which constitute the two most posterior layers of the cornea. Corneal endothelium 108 facilitates functioning of the eye. Individual endothelial cells pump water out of the cornea 104. Without them, the cornea 104 would swell from hydrostatic pressure and force fluid inside the eye into the cornea, which may result in the opacification and formation of painful blisters on the cornea. In general, the most common indication for corneal transplantation is dysfunction or loss of endothelial cells. Endothelial cells do not regenerate and replace themselves. When these cells are damaged, treatment typically involves transplantation of new endothelial cells.

### Loading or Pre-Loading the DMEK Graft Injector Assembly

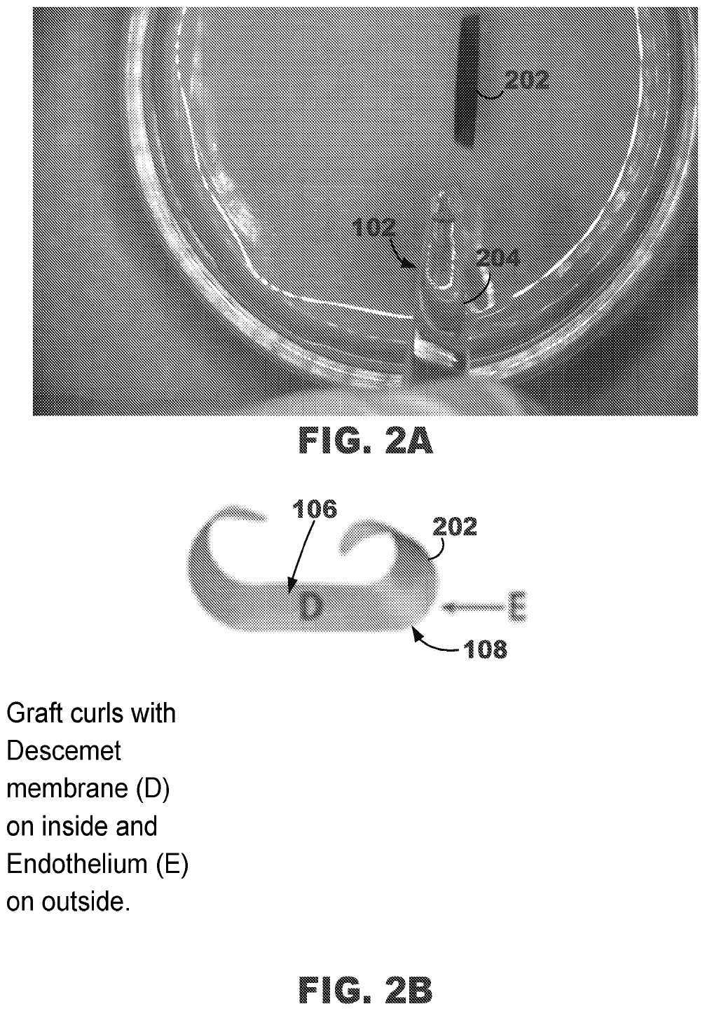

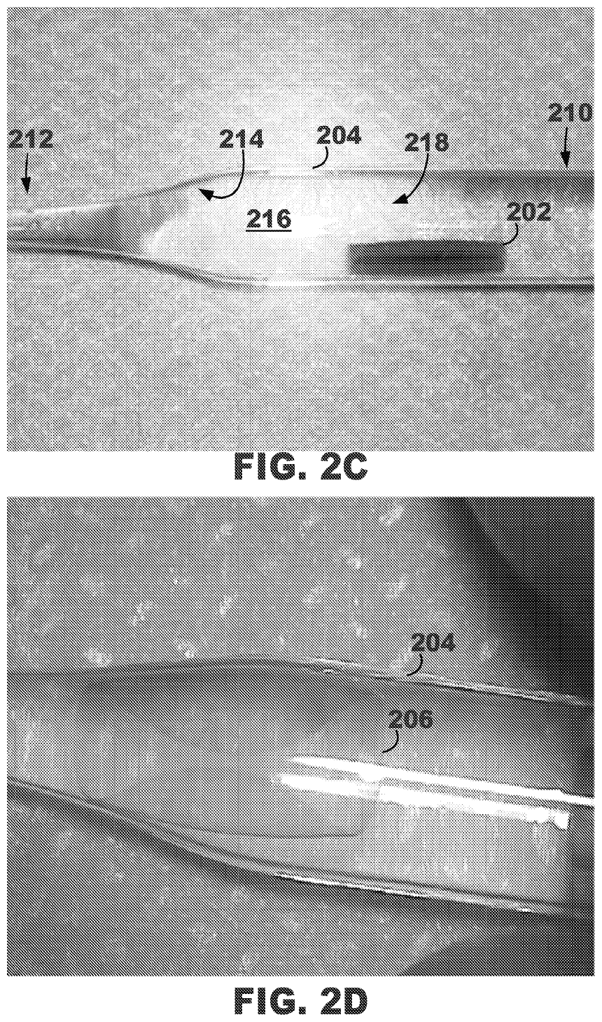

FIGS. 2A-D show one example of loading of a graft 202 into the graft injector 204 of the graft injector assembly 102. In particular, FIG. 2A shows the graft 202 outside of the graft injector 204. The graft 202 may include, or be formed of, corneal tissue or corneal cellular material. FIG. 2B shows the curling of the graft 202, which naturally curls with the Descemet membrane 106 on the inside of the curl and the endothelium 108 on the outside of the curl. FIG. 2C shows one example of the graft 202 loaded into the graft injector 204, which may be compared to FIG. 2D shows another example of a graft 206 loaded into the graft injector, which is similar to the graft 202 but is from a donor that is older in age compared to the graft 202.

In DMEK, damaged endothelium and Descemet membrane is stripped from the back of the patient's cornea 104 (FIG. 1). A healthy Descemet membrane with endothelial cells attached is then peeled off a donor cornea to form the graft 202. Because DMEK grafts are generally flimsy (for example, DMEK grafts are typically about one-fifth the thickness of a piece of paper), the graft injector 204 may be used in some implantation procedures. However, endothelial cells are very delicate and can die from any significant mechanical trauma. Furthermore, since DMEK grafts, such as graft 202 or graft 206, naturally scroll with endothelium 108 on the outside, the cells are vulnerable to damage when the graft is aspirated or ejected from the graft injector 204. During aspiration and ejection, the endothelial cells typically rub against the distal end portion, or tip, and lumen of the graft injector 204, causing cells to sheer off. A graft 206 from an older donor may be easier to transplant due to less curling (compare FIGS. 2D and 2C) but may rub against the graft injector 204 more than a younger graft 202 and be more susceptible to damage.

Some eye banks prepare and preload DMEK grafts into graft injectors and send the loaded injectors to the surgeon rather than sending the donor cornea. Using preloaded DMEK injectors saves operating time since the surgeon does not have to harvest the Descemet membrane and endothelial cells from the donor cornea. During transportation, however, the DMEK grafts can be subject to forces that cause the DMEK grafts to contact the lumen wall of the injector, which can cause damage to the vulnerable endothelial cells. Even worse, DMEK grafts can adhere to the walls of the graft injector. When that happens, as the graft is ejected, significant sheer stress can be placed on the endothelium 108 even before the graft 202 reaches the tapered tip of the injector.

In one aspect, the graft injector 204 may include, or be described as, an elongated injector body. The graft injector 204 may include a proximal end portion 210, a distal end portion 212, and an interior surface 214 extending between the proximal end portion and the distal end portion. A coating 216 may be disposed on, or applied to, the interior surface 214. A lumen 218 may be defined by the interior surface 214 and the coating 216 disposed on the interior surface. The lumen 218 may be structured to store the graft 202, or graft 206, and allow the graft to exit distally through the distal end portion 212. The coating 216, or at least one of the materials forming the coating, may reduce adhesion with the corneal tissue compared to the interior surface 214 of the graft injector 204, or at least one of the materials forming the interior surface, may increase hydrophilicity compared to the interior surface, or both.

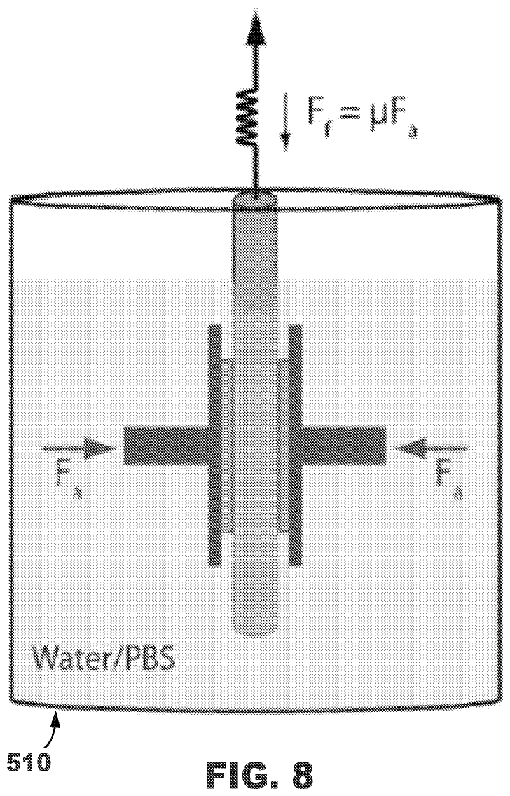

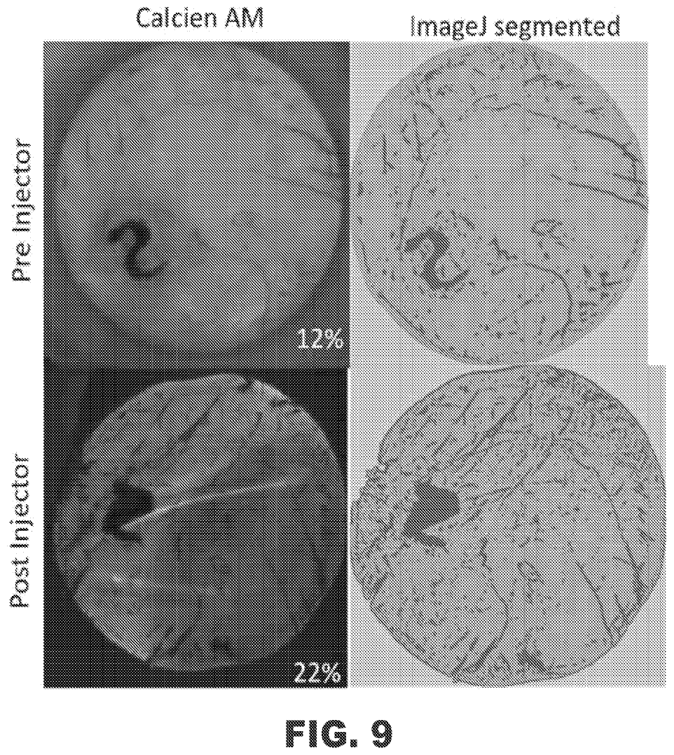

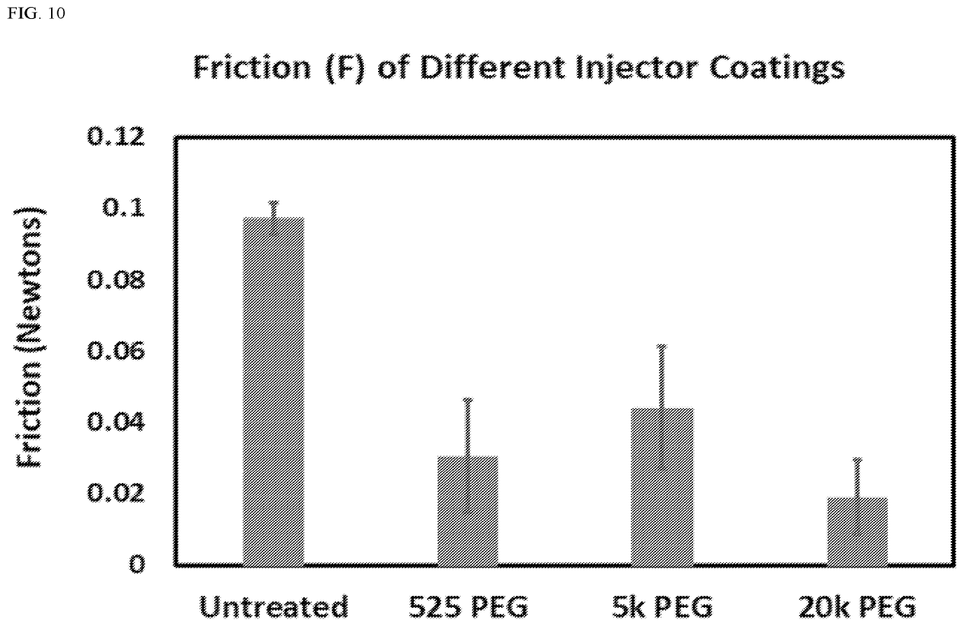

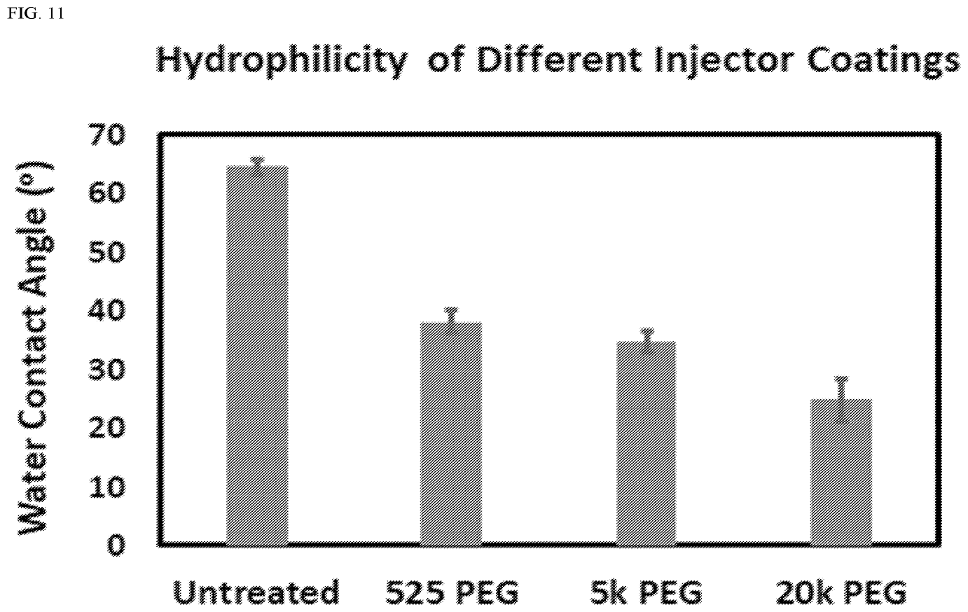

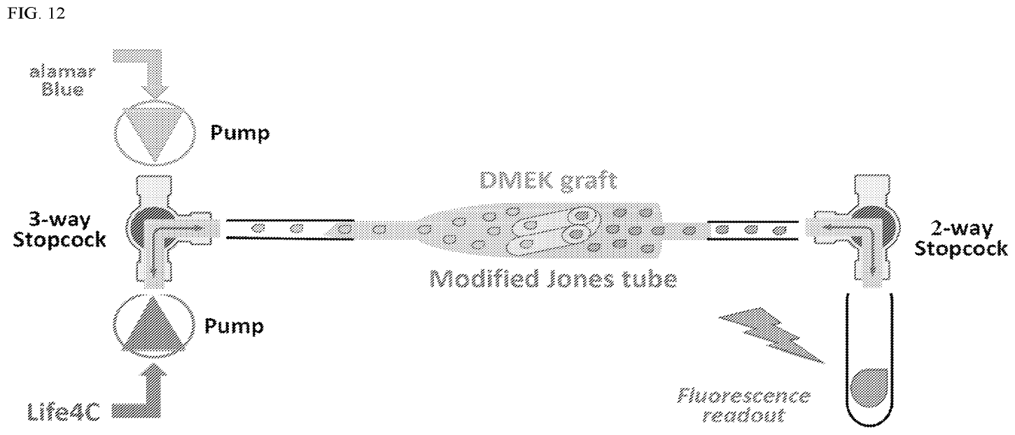

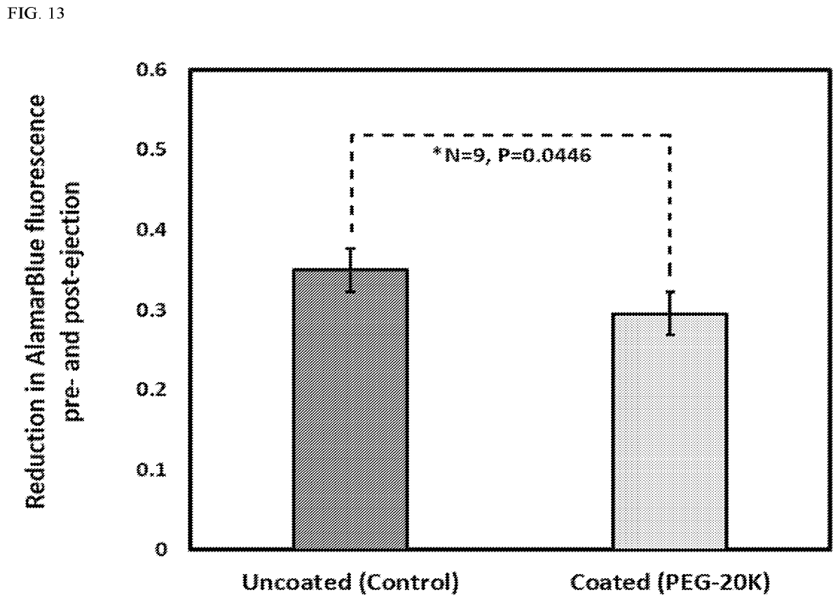

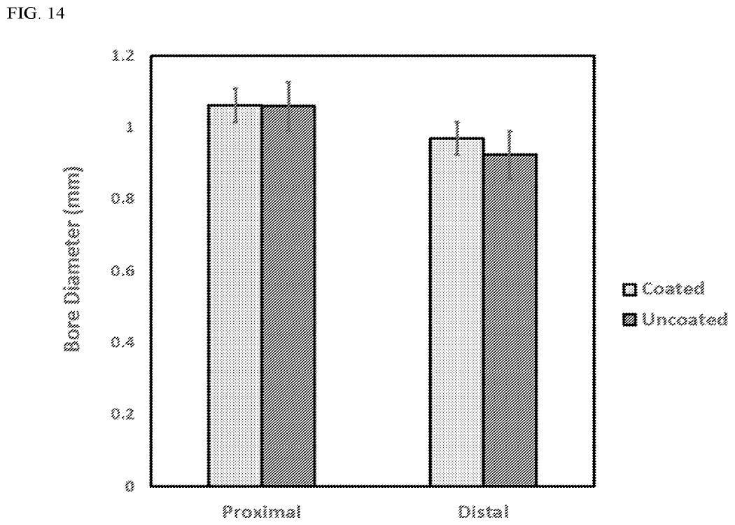

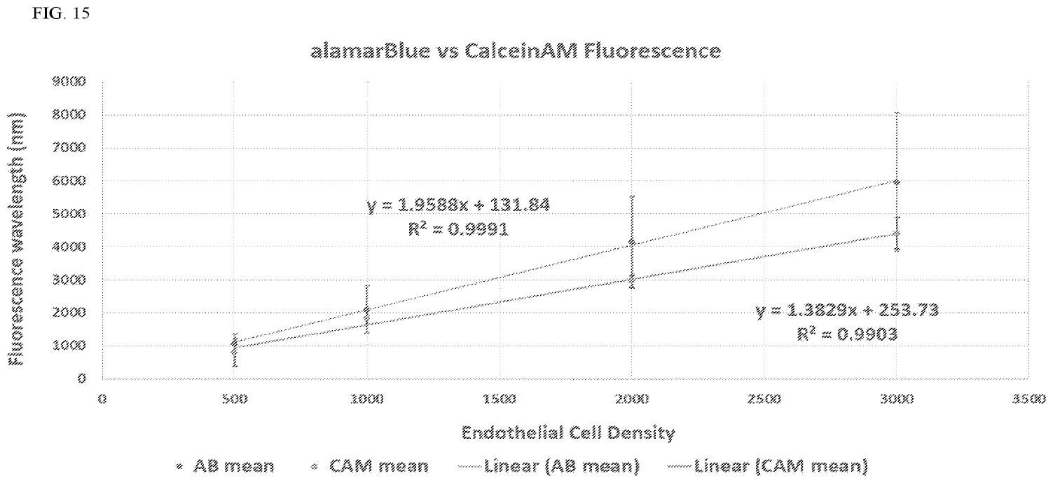

Various properties of the coating 216 may be measured to facilitate reduced adhesion of endothelial cells to the interior surface 214. Exemplary measurable properties include, but are not limited to, hydrophilicity, surface charge, chemical composition, coating texture, thickness, or durability. Hydrophilicity of the coating may be measured by recording the water contact angle using a goniometer. In general, reducing contact angle with the coating material in comparison to the uncoated surface will indicate an increase in hydrophilicity due to the coating. Lubricity of the coating can be quantified using an appropriate measurement device, such as the device illustrated in FIG. 8. Adhesion of the coating may be evaluated by a pull-out assay, where a substrate containing a monolayer of cells is dragged at a certain speed across the coated surface with which the monolayer of cells is in contact. The frictional force can be recorded and may indicate whether the coated surface provides sufficient lubricity toward the cell layer. The viability of cells may be assessed after the pull-out to determine if and which surface coating material performs well, for example, using the techniques illustrated in FIG. 9.

In some embodiments, the graft injector 204 may be formed of, or include, a glass material. In other embodiments, the graft injector 204 may be formed of, or include, a plastic material. Plastic may be more adherent to cells than glass before applying the coating 216. In general, material used to form at least part of the graft injector 204 may be visibly clear, transparent, or at least translucent, to facilitate graft evaluation and transplantation.

The coating 216 may be applied to interior surfaces of various types of graft injectors. In some embodiments, the interior surface 214 is a glass surface including, for example, a Straiko modified Jones tube (available from Gunther Weiss Scientific Glassblowing Co., Inc., Portland, OR), a pipette as included in a DMEK Disposable Surgical Set (available from DORC, the Netherlands), or a DMEK Cartridge (available from Geuder, Heidelberg, Germany).

The coating 216 may even be applied to interior surfaces of graft injectors used in graft injector assemblies. For example, the coating 216 may be applied to a graft injector used in preloaded DMEK assemblies, such as the assemblies described in U.S. Pat. No. 10,041,865, which is incorporated herein by reference.

The coating 216 may be disposed on some or all of the interior surface 214. In some embodiments, the coating 216 is disposed on the proximal end portion 210, the distal end portion 212, between the proximal and distal end portions, or any combination of these. In one embodiment, the coating 216 is disposed on the entire interior surface 214. In general, all surfaces of the graft injector 204 that may be in contact with grafts may be coated. An external, or outer, surface of the graft injector 204 handled by the surgeon or eye bank technician may remain an uncoated frictional surface for ease of handling. However, using the coating 216 on other surfaces of the graft injector 204 is also contemplated (for example, on the exterior surface).

### Coating Materials

Various materials may be used in the coating 216 to provide at least one of reduced adhesion with corneal tissue, hydrophilicity, or both. One type of material that may be used in the coating 216 is an organosilane. Organosilanes are a group of chemical compounds that have been used to create nanoparticulate coatings, for example, to enhance the lubricity and reduce friction and adhesiveness in catheters. Organosilanes may be used to provide a highly hydrophilic coating that can create a thin liquid interface on at the interface of the lumen 218 and the interior surface 214 of the graft injector 204 than can allow grafts to essentially hydroplane over the surface. Organosilane coatings can be readily applicable to glass material.

Organosilanes offer various properties that allow one to select an appropriate organosilane for a desired purpose. For example, various organosilanes provide a range of surface characteristics and can be used to alter the hydrophobicity of a coated surface. Organosilanes also can be used to covalently or non-covalently bind drugs to create drug-eluting devices. Various organosilane compositions may provide optimal surface characteristics, including compositions that provide minimal glass-to-tissue friction and compositions that provide minimal glass-to-tissue adhesion. Using a coated glass injector with high hydrophilicity and lubricity may minimize contact-induced endothelial cell damage in DMEK by reducing cell-to-glass friction and adhesion. This, in turn, can improve the safety and outcome of DMEK surgery overall for patients receiving either surgeon prepared or preloaded DMEK grafts. The coating may be further modified to elute an antibacterial agent or an antifungal agent. For example, the coating may be modified to elute amphotericin B to reduce the risk of fungal keratitis from preloaded DMEK.

In general, any functional bioactive moiety may be used in the coating 216 of a glass surface of the graft injector 204 for the purpose of achieving enhanced lubricity and tissue compatibility. Non-limiting examples of such functional bioactive moieties include amine, carboxylic acid, hydroxyl, sugar and polysaccharide, glycosaminoglycan (GAG), peptide, protein, polymer, or any combinations of these. Polymers may have various molecular sizes, chain lengths, or other architecture. Various examples of polymer include but are not limited to a polyethylene glycol (PEG), a polyvinyl alcohol, a polyhydroxy ethyl methacrylate (pHEMA), a poly(meth)acrylic acid (PMAA), a polyvinylpyrrolidone, or any combination of these. In one or more embodiments, the coating 216 includes amine, polyethylene glycol, or both.

### Coating Methods

In another aspect, a method of forming a graft injector with an interior coating includes bonding a coating to an interior surface of an elongated injector body. The method also includes attaching a functional bioactive moiety to the coating to reduce adhesion to a graft having corneal tissue compared to adhesion of the uncoated interior surface with the graft.

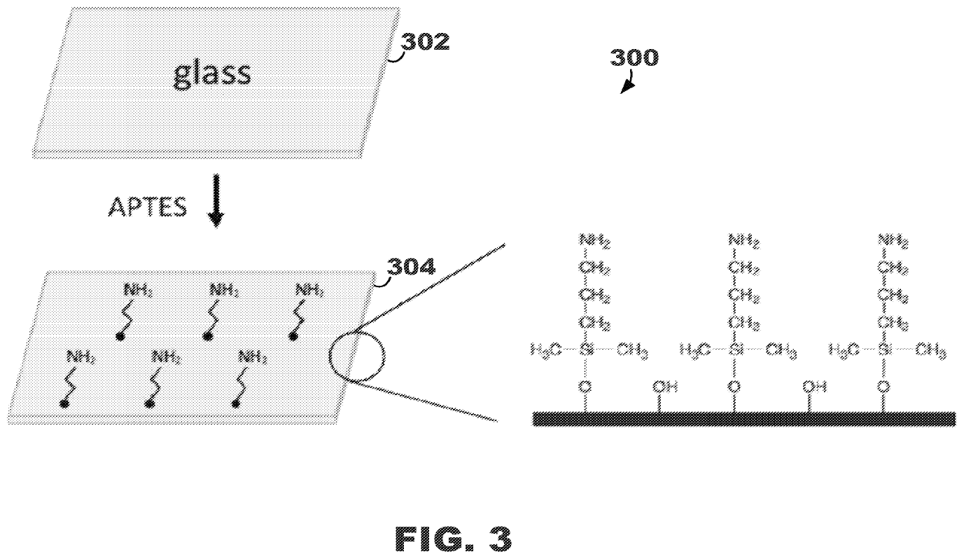

FIG. 3 shows one example of a coating method 300 used to form a coating on a glass graft injector. The method 300 may include applying 3-aminopropyl triethyoxysilane (APTES) to the glass surface 302 to form a coated surface 304. In the illustrated embodiment, the coating is covalently bonded to the glass surface of the injector. The coated surface 304 formed may reduce friction between the interior surface of the injector and corneal tissue, such as endothelium.

In the illustrated embodiment, coated surface 304 contains amine functional groups and polyethylene glycol (PEG) functional groups of various chain lengths that are combined in different ratios to minimize cell adhesion and friction against the lumen wall of the injector. In general, positively charged amine groups may enhance the hydrophilicity of the injector surface, thus minimizing friction by forming a layer of water at the glass-tissue interface. The charge-neutral PEG groups may reduce friction and prevent cell adhesion.

The purpose of the coated surface 304 is to prevent endothelial cell death from friction and adhesion of the DMEK graft to the walls of the injector. The resulting injector assembly having the coated surface 304 can be used by surgeons performing DMEK corneal transplantation surgery or by eye banks that preload the DMEK grafts and ship the DMEK grafts to surgeons already loaded in the injector. The coated glass injector may provide superior protection of the DMEK graft during loading (in the eye bank or in the operating room (OR)), transportation (from eye bank to OR), and insertion (in the OR) of the graft. The assembly may be designed so the coating is on all surfaces where the cells of the graft may come into contact with the injector surface. This may include the lumen surface of the injector and the tip. The coating on the tip may facilitate insertion of the injector into the eye through a standard surgical wound.

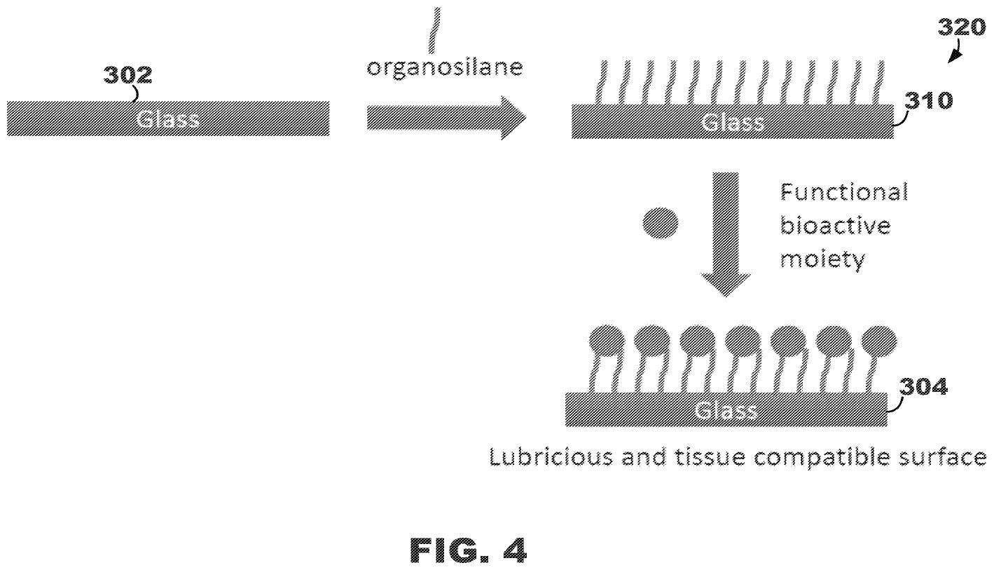

FIG. 4 shows another example of a coating method 320 used to form a coating on a glass graft injector including a functional bioactive moiety. The method 320 may be similar to the method 300 except that method 320 may include an intermediate step of attaching one or more functional bioactive moieties to an organosilane-coated surface 310 to form the final coated surface 304.

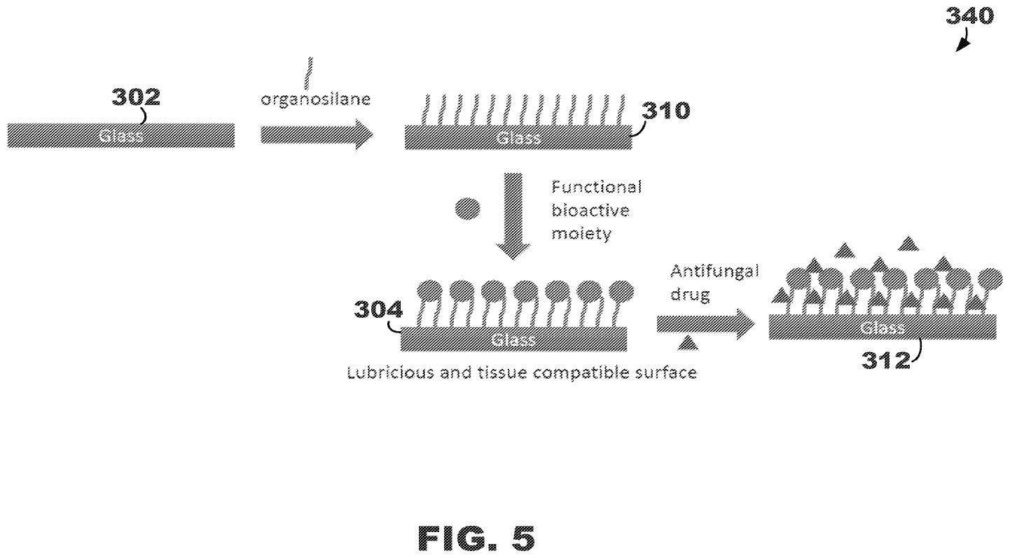

FIG. 5 shows yet another example of a coating method 340 used to form a coating on a glass graft injector including an antimicrobial agent. The method 340 may be similar to the method 320 except that method 340 may include an additional step of attaching one or more antimicrobial drugs, or other antimicrobial agent, to the coated surface 304 to form an antimicrobial coated surface 312. Although illustrated in the context of an exemplary embodiment in which the antimicrobial agent is an antifungal agent, the antimicrobial agent may alternatively or additionally include an antibacterial agent.

The antimicrobial coated surface 312 may be used in a drug-eluting coated graft injector. The antimicrobial coated surface 312 may incorporate an antimicrobial drug onto the surface via physical entrapment, chemical conjugation, immobilization of nano- or micro-scale drug carriers, or any combination of these. In some embodiments, the antimicrobial agent may be non-covalently bonded to an organosilane coating and released once the graft injector is submerged in solution. The antimicrobial coated surface 312 may provide long-lasting antimicrobial activity by the sustained elution and delivery of otherwise unstable (for example, short half-life in solution) anti-fungal medications that can help to maintain the sterility of DMEK grafts during transportation. Any suitable antimicrobial drug may be used, such as, for example, a polyene, an azole, an allylamine, an echinocandin, an antibiotic, or combinations of two or more antimicrobial drugs. Non-limiting examples of antimicrobial drugs include amphotericin B micelles (a polyene antifungal) and voriconazole (an azole antifungal). Some antifungal drugs that may be used may also be described as “antifungal drug X” micelles.

In another aspect, a method for forming a loaded or preloaded DMEK graft injector assembly includes bonding a coating to an interior surface of a graft injector to define a lumen by the coating and the interior surface, loading a solution into the lumen of the graft injector, and loading a graft into the lumen of the graft injector.

Using a DMEK Graft Injector Assembly with Interior Coating

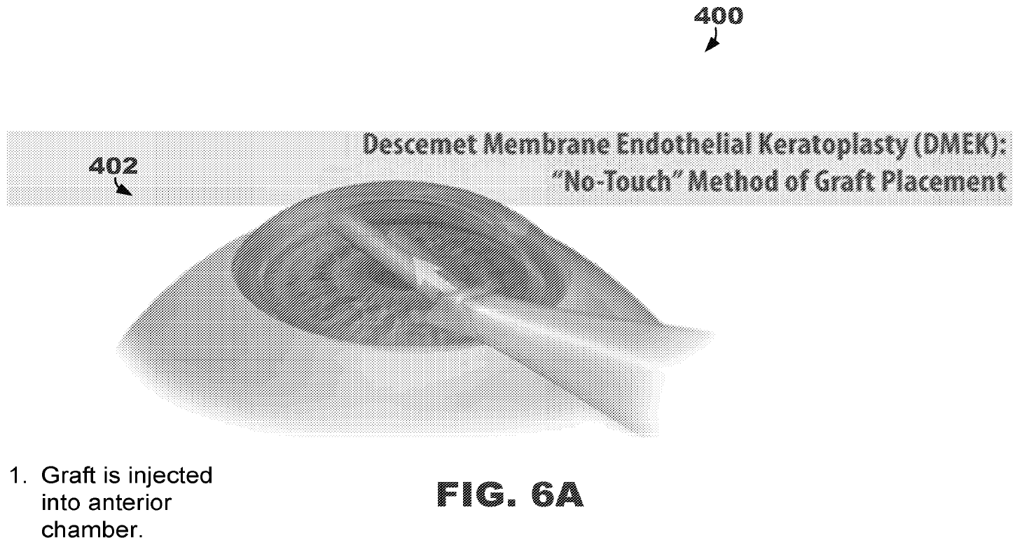

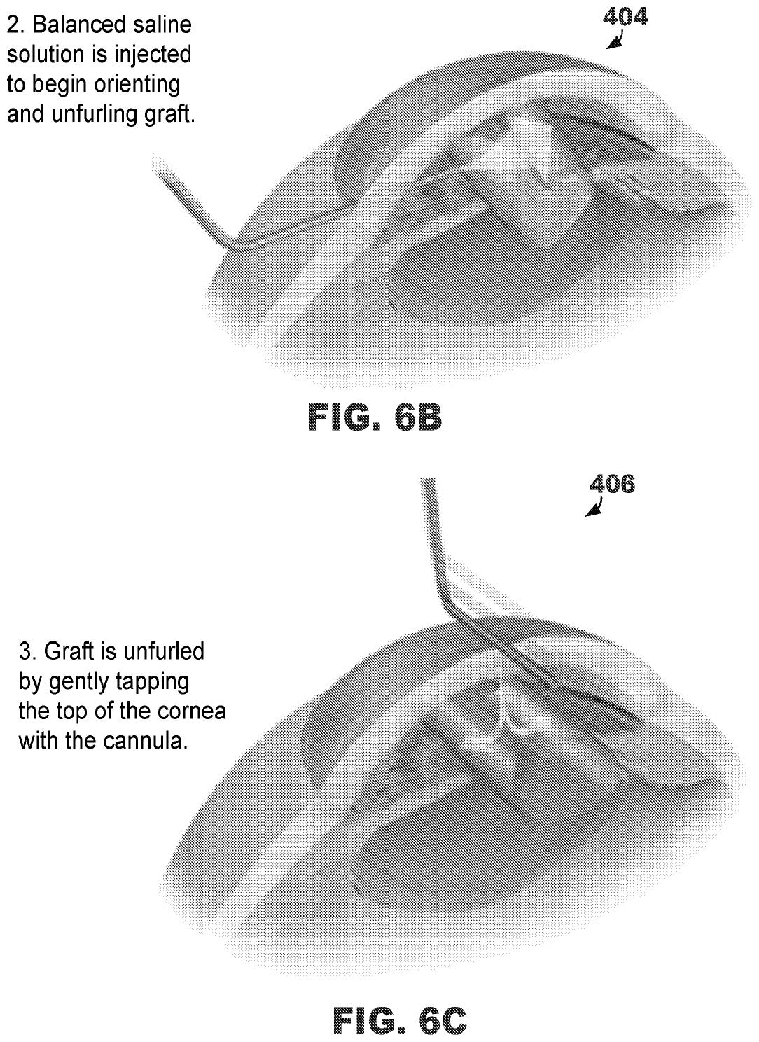

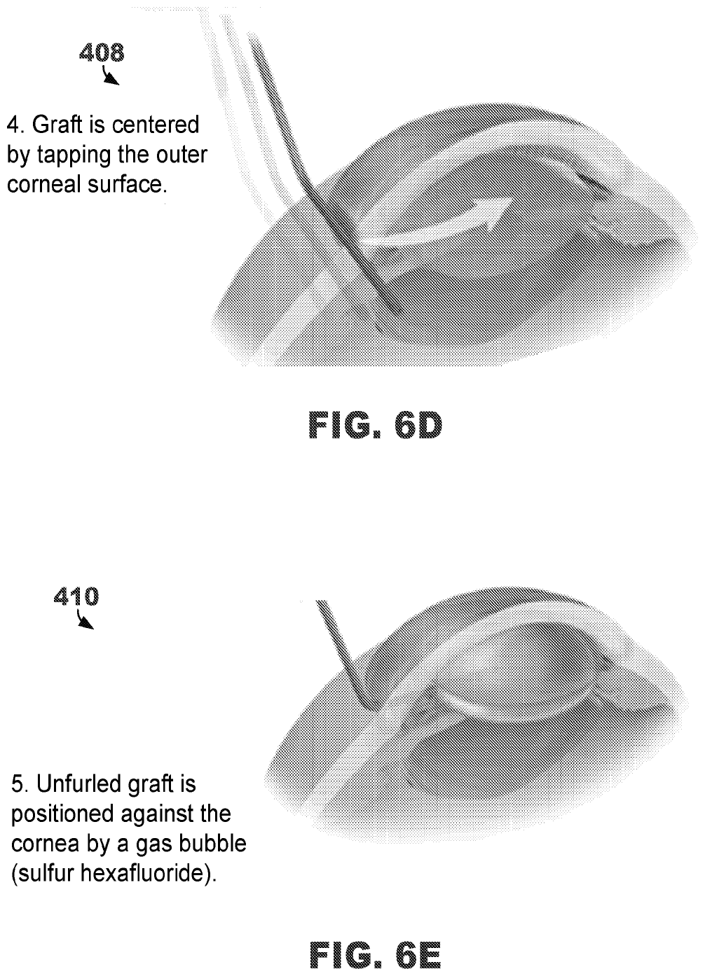

FIGS. 6A-E show one example of a method 400 of placing a graft using the graft injector assembly in Descemet membrane endothelial keratoplasty (DMEK). FIG. 6A shows a process 402 of injecting a graft into the anterior chamber of the patient's eye. The coated DMEK assembly may be used to carry out the injection. After injecting the graft into the eye, in general, the graft is unscrolled and centered without any further touching of the graft, for example, using jets of fluid or external tapping. FIG. 6B shows a process 404 of injecting balanced saline solution into the patient's eye. The jets of fluid created by injecting the solution may facilitate orienting and unfurling the graft. FIG. 6C shows a process 406 of tapping the cornea with a cannula, which may also have been used to inject the balanced saline solution. The tapping motion may facilitate further unfurling of the graft. FIG. 6D shows a process 408 of tapping a side of the corneal surface of the patient's eye with the cannula. The tapping motion may facilitate centering of the graft in the patient's eye.

Once the graft is appropriately positioned, an air or other gas bubble may be used to position the graft against the recipient cornea. FIG. 6E shows a process 410 of injecting a gas bubble containing sulfur hexafluoride below the graft. The bubble may position the graft against the cornea.

The DMEK graft may adhere to the recipient cornea after about 24 to 48 hours and the bubble of air or other gas may dissipate. In order for the surgery to be successful, a sufficient number of the endothelial cells on the DMEK graft must survive the transplantation. The overall number of cells that survive may strongly determine how quickly the graft will start working to clear the vision and how long the graft will last before failure. Even after surgery, the endothelial cells are expected to die slowly over time. Thus, the likelihood of a positive patient outcome is increased if the transplant successfully delivers a sufficient population of viable endothelial cells.

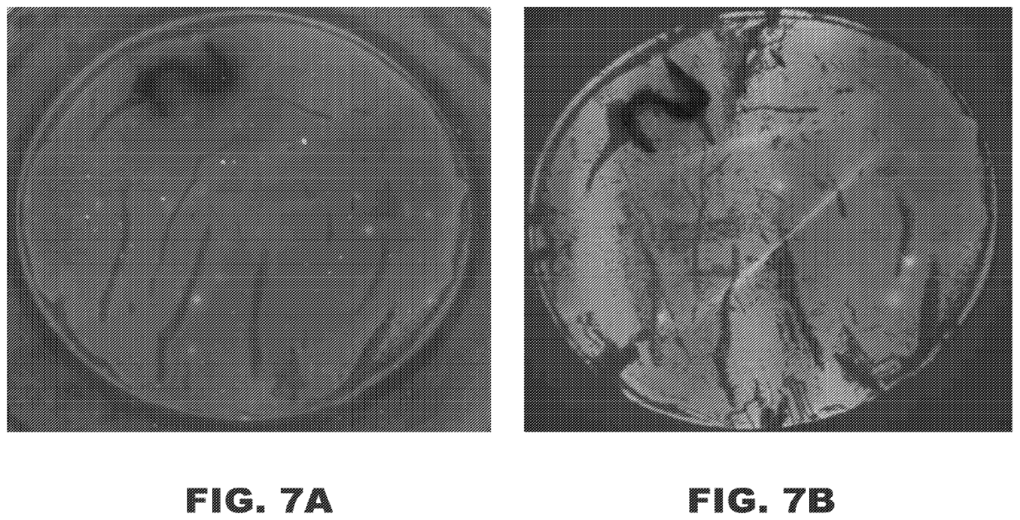

FIGS. 7A-B are staining images that illustrate (A) a graft before loading and (B) a graft after loading and ejection from a graft injector without a coating. In the illustrated embodiment, the green stain used is calcein acetoxymethyl ester (calcein AM). Calcein AM is a vital dye that stains live endothelial cells on the graft. Black areas without any green stain indicate where endothelial cell loss has occurred. In comparing the image of FIG. 7A and the image of FIG. 7B, one can see areas in FIG. 7B where endothelial cells were lost during loading and ejection from a graft ejector.

Extrapolating from mathematical models of endothelial cell loss over time following full-thickness corneal transplantation (penetrating keratoplasty), baseline endothelial cell density at transplantation affects long-term survival of the graft. For example, a graft that has 3500 endothelial cells/mm² at baseline, is expected to survive approximately 38 years; a graft that has 3000 endothelial cells/mm² at baseline is expected to survive 33 years; a graft that has 2500 endothelial cells/mm² is expected to survive 28 years; a graft that has 2000 endothelial cells/mm² is expected to survive 21 years; and a graft that has only 1500 endothelial cells/mm² is expected to only survive 12 years. In general, the threshold for failure is considered to be when the graft falls below 500 endothelial cells/mm². Thus, baseline cell density is an important predictor of graft survival.

Using methods that increase the baseline cell density after transplantation when starting with a given donor cell density therefore improves the likelihood of a successful transplant and/or the effective survival of a transplant. For example, a donor graft with a cell density of 2000 endothelial cells/mm² can lose, for example, 38.0% cell density when using an uncoated injector, leaving the transplanted graft with a baseline cell density of about 1240 endothelial cells/mm². Instead, using a coated injector may decrease endothelial cell loss to 29.5% so that the donor graft of 2000 endothelial cells/mm² produces a baseline cell density of approximately 1410 endothelial cells/mm² after transplantation. The difference in baseline endothelial cell density when using a coated injector can translate to several additional years of graft survival.

Although injector-induced injury is not exactly the same as simply starting with a graft that has lower endothelial cell density, it provides a conceptual framework for understanding the impact of having dramatic cell loss at the time of transplant. The effect of endothelial cell loss is greater for grafts with lower starting endothelial cell density. Grafts with greater starting endothelial cell density tolerate injury at the time of surgery better. However, reducing that injury in any way can result in a meaningful extension of the lifetime of any graft.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

For any method disclosed herein that includes discrete steps, the steps may be conducted in any feasible order. And, as appropriate, any combination of two or more steps may be conducted simultaneously.

As used herein, the terms “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

The present invention is illustrated by the following examples. It is to be understood that the particular examples, materials, amounts, and procedures are to be interpreted broadly in accordance with the scope and spirit of the invention as set forth herein.

### EXAMPLES

In various examples, the potential benefit of using a coated glass injector for preload DMEK in order to minimize ECL during processing and transportation will be evaluated in an experiment. In Example 1, the lubricity and hydrophilicity of glass modified with organosilane functional groups will be compared. Friction and adhesion between flattened corneal tissue and glass treated with organosilane containing amine or polyethylene glycol (PEG) of various chain lengths will be accessed to evaluate lubricity. In Example 2, ECL during loading and transportation of preloaded DMEK grafts using an uncoated glass tube injector versus a coated glass tube injector will be compared. ECL will be evaluated with live/dead vital dye staining after loading, simulated transportation, and ejection of DMEK grafts. In Example 3, the further modification of a glass tube injector by noncovalently binding of amphotericin B micelles to the organosilane functional group will be evaluated. Evaluation of the amphotericin concentration into standard corneal storage medium from a modified Jones tube will be measured over time.

### Example 1

Example 1 compares the lubricity of glass modified with organosilane containing amine or PEG of various chain lengths and determine the optimal modification to minimize friction. At least two different surface coating strategies will be explored. First, introduction of positively charged amine groups is expected to enhance the hydrophilicity of glass surface, thus minimizing friction by forming a layer of water at the glass/tissue interface (see FIG. 3). Second, charge-neutral surface coating of glass will be created using PEG silanes, which is expected to not only reduce friction but also prevent adhesion of endothelial tissue (see FIG. 3). The thickness of the surface coating will be tuned conveniently by using PEG silanes of various molecular weights. Further, both amine and PEG will be introduced into the coating simultaneously, and the ratio of the two will be optimized to achieve the best enhancement of lubricity and prevention of tissue adhesion.

### Synthesis and Characterization of Organosilane Coatings

The chemical reaction will first be conducted with flat glass slides to allow easy characterization and then applied to modified Jones tubes. To prepare amine-coated glass, (3-aminopropyl)triethoxysilane (APTES) (available from Gelest, Inc., Morrisville, Pennsylvania) will be dissolved in ethanol/H₂O (95/5%, pH adjusted to 5 with acetic acid) to a final concentration of 2% by weight (wt. %). Glass surface will be dipped in the silane solution for 2-3 minutes before washing with ethanol twice. The coating will be cured at room temperature for 24 hours before characterization. The same procedure will be used to react 2-[methoxy(polyethyleneoxy) propyl]trimethoxysilane (PEGn-TMS, n=6-9, 9-12, 21-24) of various chain length (available from Gelest, Inc., Morrisville, Pennsylvania) to generate PEG coating or mixed coating using mixtures of APTES and PEGn-TMS.

Coated glass surfaces will be characterized by static water contact angle measurement using a goniometer. It is expected that enhanced hydrophilicity would result in decrease in water contact angle in comparison to unmodified glass (approx. 25 degrees).

### Friction Assay

Lubricity of modified glass surfaces will be quantified using a customized apparatus 510 at the University of Minnesota Characterization Facility as shown in FIG. 8. Frictional force (Fr) is recorded as the surface, immerged in phosphate buffered saline (PBS) mimicking storage media, is compressed (Fₐ) to determine the friction coefficient (μ) of that particular surface. Frictional force (F_{f}) against flattened DSAEK tissue mounted with endothelium facing in at the compression interface will also be measured to assess frictional forces against endothelial tissue. An optimal modified surface with the highest lubricity for studies may be identified in Examples 2 and 3.

### Example 2

Example 2 compares ECL during loading and transportation of preloaded DMEK grafts using an uncoated glass tube injector versus a coated glass tube injector. Preliminary data has demonstrated an average of 14.5% more ECL with preloaded DMEK compared to pre-trephinated DMEK (p=0.011, 2-tailed paired t-test). Having demonstrated a significant increase in ECL with preloaded DMEK, the efficacy of using a coated glass injector over a standard glass injector using similar protocols will be tested.

### Sample Size

Based on preliminary data, mean ECL with preload DMEK in our eye bank was 24.5% (standard deviation 6.6%). In order to detect a 50% improvement in ECL with use of a coated injector (at least a 7.25% difference in ECL between using a coated and uncoated injector), a sample size of 30 will be targeted in order to achieve sufficient statistical power (α=0.05, β=0.80).

### Preparing DMEK Grafts

Standard SCUBA peel will be performed on mated donor cornea pairs. No “S” stamp will be applied to avoid confounding factors for ECL. Peeled corneas will be trephinated with a 7.75 mm corneal trephine, then stained for 30 minutes with 2 μM calcien acetoxymethyl ester (calcien AM) in Optisol-GS storage medium (available from Bausch and Lomb, Rochester, NY) to assess baseline endothelial cell death.

### Endothelial Cell Loss After Preloading

One cornea from each mated pair will be randomized to preloading with a standard Straiko Modified Jones Tube (80000-DMEK-EB) (available from Gunther Weiss Scientific, Inc., Portland, OR). The other cornea from each mated pair will be randomized to preloading with an organosilane coated Straiko Modified Jones Tube. The preloaded DMEK scrolls will be evaluated at slit lamp to document any areas of adhesion or graft-glass touch. The DMEK grafts will then be ejected onto teflon dish with fresh calcein acetoxymethyl ester (calcein AM) and unrolled carefully to avoid damage. Repeat fluorescence imaging will be obtained and compared to baseline images to determine total ECL. Additionally, unloaded DMEK scrolls of similar donor age will be unrolled onto a teflon dish immediately after peeling and used as age-matched controls to account for potential damage during the unrolling process itself. Significantly less endothelial cell loss with preloading in the nano-coated glass injector is expected.

Endothelial Cell Loss after Transportation

Additional DMEK grafts will be prepared from mated donor corneas. Grafts from each mated pair will be randomized to loading in either an uncoated or a coated injector. The preloaded grafts will then be shipped in a standard cornea viewing chamber (CVC) to a local surgicenter, stored overnight, and then shipped back. The grafts will then be ejected, unscrolled, and imaged as described above to assess total ECL from preloading and transportation. Significantly less endothelial cell loss with transportation in the nano-coated glass injector is expected.

### Imaging Endothelial Cell Loss

ECL will be evaluated using a calcein acetoxymethyl ester (Calcein AM) viability assay. Fluorescence imaging will be taken with a Nikon DS-Fi3 camera mounted on Nikon SMZ1500 microscope with P-EFL fluorescence attachment loaded with GFP light filter set from single field (no stitching) to produces raw images after staining the cells, similar to images shown in FIG. 9. The images will be segmented into live, dead, and background areas with ImageJ Weka Segmentation plug-in (available from National Institutes of Health, Rockville, MD), similar to images as shown in FIG. 9. Areas of ECL will be calculated from the number of pixels in dead/(live+dead) segments. The difference in total ECL after preloading or transportation compared to baseline will be calculated to determine overall efficacy of using a coated injector.

### Example 3

Example 3 evaluates the potential for non-covalently binding of amphotericin B micelles to a coated glass injector and measure elution of the drug over time in storage media, which may identify the potential for further modification of organosilane functional groups to non-covalently bond amphotericin B micelles and provide slow elution of the antimycotic into solution.

### Binding Amphotericin Micelles to Glass Surface and a Coated Jones Tube

Given that amphotericin is insoluble in water, the drug in block copolymer micelles consisting of a PEG-phospholipid will be encapsulated. The drug-encapsulated micelles will be dispersed in organosilane aqueous solution and dip coated onto glass surface. Higher dose of drug coating may be achieved by prolonging the dip-coating process. Coated glass surfaces will be characterized by water contact angle measurement and lubricity testing described above. In the unlikely case that drug-containing micelles affected the lubricity coating, a separate top layer coating without micelles may be applied.

### Assessing Amphotericin B Concentration

A CVC with a coated, eluting Jones tube in Optisol-GS will be monitor in cold storage at 4 degrees Celsius for 48 hours. Serial samples of the Optisol solution will be obtained from the CVC at 3, 6, 12, 24, and 48 hours after insertion of the modified injector. Samples will be evaluated for amphotericin B concentration in solution using spectrophotometric analysis (absorbance at 325 nanometers).

### Example 4

Two sets of Weiss Glass DMEK injectors (LEITR tubes) were prepared. One set was coated with PEG-20K to reduce surface friction and increase lubricity/hydrophilicity. The other set was left uncoated as a control. Donor pairs of research corneas, identified as suitable for DMEK, were peeled then loaded into coated and uncoated tubes. Endothelial cell viability was then assessed using an ALAMARBLUE assay (Trek Diagnostic Systems LLC, Cleveland, OH), which elicits a fluorescence signal that is proportional to total cell metabolism and viability in a tissue sample and can be used to measure cell viability in DMEK grafts both inside and outside the injector. Both coated and uncoated LEITR tubes containing a single DMEK graft, were then individually attached to a peristaltic pump for the addition of ALAMARBLUE reagent. The tubes were disconnected from the pump, sealed at both ends, then incubated for one hour at 37° C. to allow the conversion of the ALAMARBLUE reagent into a highly fluorescent derivative by viable endothelial cells on the DMEK graft. After the first incubation period, the tubes were reconnected to the peristaltic pump for collection of the fluorescent sample. Once the collection was complete, the DMEK grafts were ejected from the glass tube into a well containing fresh ALAMARBLUE reagent. A second hour-long incubation at 37° ° C. ensued with the post-ejection fluorescent samples collected at the end. The fluorescence intensity of the collected samples was analyzed using a microplate reader (BioTek Synergy, Agilent Technologies, Inc., Santa Clara, CA).

All scientific and technical terms used herein have meanings commonly used in the art unless otherwise specified. The definitions provided herein are to facilitate understanding of certain terms used frequently herein and are not meant to limit the scope of the present disclosure.

The phrases “at least one of,” “comprises at least one of,” and “one or more of” followed by a list refers to any one of the items in the list and any combination of two or more items in the list.

1. A graft injector assembly comprising:
an elongated injector body formed from at least a first material, the body comprising a proximal end portion, a distal end portion, and an interior surface extending between the proximal end portion and the distal end portion; and
a coating formed from at least a second material disposed on the interior surface of the body, wherein a lumen is defined by the interior surface and the coating, the lumen structured to store a graft comprising corneal tissue and to allow the graft to exit distally through the distal end portion.

2. The assembly according to claim 1, wherein adhesion of the second material to corneal tissue is less than adhesion of the first material to corneal tissue.

3. The assembly according to claim 1, wherein the second material is more hydrophilic than the first material.

4. The assembly according to claim 1, further comprising the corneal graft, the corneal graft comprising a Descemet membrane and endothelial cells.

5. The assembly according to claim 1, wherein the first material comprises a glass material.

6. The assembly according to claim 1, wherein the coating is disposed on the entire interior surface of the body.

7. The assembly according to claim 1, wherein the coating is covalently bonded to the body.

8. The assembly according to claim 1, wherein the second material comprises a positively charged functional group, a charge-neutral functional group, or both.

9. The assembly according to claim 1, wherein the second material comprises an organosilane material.

10. The assembly according to claim 1, wherein the second material comprises a nanoparticulate material.

11. The assembly according to claim 1, wherein the second material comprises a functional bioactive moiety compatible with the graft that lubricates the interior surface.

12. The assembly according to claim 11, wherein the functional bioactive moiety is selected from amine, carboxylic acid, hydroxyl, sugar, polysaccharide, glycosaminoglycan, peptide, protein, polymer, or any combination of these.

13. The assembly according to claim 12, wherein the polymer is selected from a polyethylene glycol, a polyvinyl alcohol, a polyhydroxy ethyl methacrylate, a poly(meth)acrylic acid, a polyvinylpyrrolidone, or any combination of these.

14. The assembly according to claim 1, wherein the second material comprises a polymer comprising amine, polyethylene glycol, or both.

15. The assembly according to claim 1, wherein the second material comprises polymers of various chain lengths.

16. The assembly according to claim 1, wherein the coating further comprises an antifungal agent.

17. The assembly according to claim 16, wherein the antifungal agent is non-covalently bonded to the second material.

18. The assembly according to claim 16, wherein the antifungal agent is coupled to the second material using physical entrapment, chemical conjugation, immobilization, or any combination of these.

19. A method comprising:
bonding a coating to an interior surface of a graft injector to define a lumen by the coating and the interior surface;
loading a solution into the lumen of the graft injector; and
loading a graft into the lumen of the graft injector.

20. A method comprising:
bonding a coating to an interior surface of an elongated injector body; and
attaching a functional bioactive moiety to the coating to reduce adhesion to a graft comprising corneal tissue compared to adhesion of the interior surface with the graft.
